(19) United States
(12) Patent Application Publication
(10) Pub. No.: US 2025/0221992 A1
(43) Pub. Date: Jul. 10, 2025

(54) Methods Of Treating Depression Using Orexin-2 Receptor Antagonists

(71) Applicant: Janssen Pharmaceutica NV, Beerse (BE)

(72) Inventors: Peter De Boer, Breda (NL); Justine M. Kent, Titusville, NJ (US); Wayne C. Drevets, Titusville, NJ (US)

(21) Appl. No.: 19/016,620

(22) Filed: Jan. 10, 2025

Related U.S. Application Data
(63) Continuation of application No. 17/551,955, filed on Dec. 15, 2021, now Pat. No. 12,201,634.
Application No. 17/551,955 is a continuation of application No. 17/092,725, filed on Nov. 9, 2020, now Pat. No. 11,241,432.
Application No. 17/092,725 is a continuation of application No. 15/454,628, filed on Mar. 9, 2017, now Pat. No. 10,828,302.
(60) Provisional application No. 62/306,487, filed on Mar. 10, 2016.

(51) Int. Cl. A61K 31/506; A61K 9/00; A61K 9/10; A61K 9/20; A61K 31/50; A61K 31/53; A61K 45/06; A61K 47/38

(52) U.S. Cl. CPC A61K 31/506; A61K 9/0095; A61K 9/10; A61K 9/2018; A61K 9/2027; A61K 45/06; A61K 47/38; A61K 31/50; A61K 31/53

(57) ABSTRACT

The present disclosure is directed to, inter alia, methods of treating a subject suffering from or diagnosed with depression, comprising administering to a subject in need of such treatment an effective amount of a compound of formula (I), or a pharmaceutically acceptable salt thereof, wherein R¹ to R⁴ are described herein and wherein the compound is administered prior to sleep.

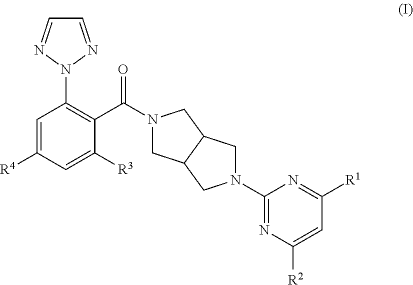

### CROSS-REFERENCE TO RELATED APPLICATIONS

This application is a continuation of U.S. patent application Ser. No. 17/551,955, filed Dec. 15, 2021, which is a continuation of U.S. patent application Ser. No. 17/092,725, filed Nov. 9, 2020, which is a continuation of 15/454,628, filed Mar. 9, 2017, now U.S. Pat. No. 10,828,302, issued on Nov. 10, 2020, which claims priority to U.S. Provisional Patent Application No. 62/306,487, filed Mar. 10, 2016, all of which are incorporated by reference herein.

### TECHNICAL FIELD

The present disclosure is directed to, among other things, methods for the treatment of depression.

### BACKGROUND

Orexins (also known as hypocretins) are neuropeptides expressed by neurons in the perifornical area, the dorsomedial hypothalamus and the lateral hypothalamus (de Lecea et al., 1998; Proc. Natl. Acad. Sci. U.S.A. 95, 322-327; Sakaurai et al, 1998, Cell 92, 573-585). Orexinergic neurons project to many areas of the brain including other hypothalamic nuclei, the midline paraventricular thalamus, brain stem nuclei, the ventral tegmental area and nucleus accumbens shell. (Peyron et al., 1998, J. Neurosci. 18, 9996-10016). Orexin neuropeptides, classified as either orexin-A or orexin-B, bind to the seven transmembrane G-protein coupled receptors orexin-1 (OX1R) and orexin-2 (OX2R) (de Lecea et al., 1998; Proc. Natl. Acad. Sci. U.S.A. 95, 322-327; Sakaurai et al, 1998, Cell 92, 573-585). While orexin-A is non-selective for OX1R and OX2R, orexin-B shows higher affinity for OX2R (Sakaurai et al, 1998, Cell 92, 573-585). Orexin receptor antagonists are classified as single orexin receptor (SORAs) or dual receptor antagonists (DORAs).

Hypothalamic orexinergic neurons expressing discharge during active wake, are virtually silent during non-rapid eye movement sleep and show transient discharges during rapid eye movement sleep (Lee, 2005, J. Neuroscience 25(8): 6716-6720; Takahashi, 2008, Neuroscience, 153: 860-870). This activity pattern supports the notion that the orexins are endogenous, potent, arousal (wakefulness)-promoting peptides. Studies using single unit recordings also show that OX-containing neurons are preferentially activated during rewarding appetitive behaviors (Hassani et al., 2016. J Neuroscience 36(5): 1747-1757). However, orexins are also hypothesized to play a role in excessive arousal (e.g. hypervigilance, anxiety, somatic tension, agitation and/or excessive rumination) which occurs in subsets of patients with mood disorders. To date, it is believed that intrinsic antidepressant activity of a selective OXR2 antagonist has not been explored clinically.

As is known in the art, clinically significant improvement in symptoms of depression in subjects diagnosed with Major Depressive Disorder (MDD) may take 4-6 weeks after the initiation of treatment with currently available antidepressants. Therefore, it is not expected that MDD subjects would benefit from shorter periods of antidepressant therapy, especially 2 weeks or less. There remains a high, unmet medical need to provide an effective treatment for depression.

### SUMMARY

The general description and the following detailed description are exemplary and explanatory only and are not restrictive of the disclosure, as defined in the appended claims. Other aspects of the present disclosure will be apparent to those skilled in the art in view of the detailed description of the disclosure as provided herein.

In one aspect, methods of treating a subject suffering from or diagnosed with depression are provided. These methods comprise administering to a subject in need of such treatment an effective amount of a compound of formula (I), or a pharmaceutically acceptable salt thereof, wherein R¹-R⁴ are defined herein, and wherein the compound of formula (I) is administered prior to sleep.

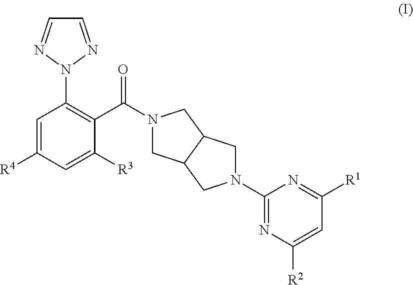

In another aspect, the subject treated according to the methods described herein is not suffering from or diagnosed with an insomnia disorder.

In a further aspect, the compound of formula (I) is administered at night according to the methods described herein.

### BRIEF DESCRIPTION OF THE FIGURES

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The summary, as well as the following detailed description, is further understood when read in conjunction with the appended figures:

FIGS. 1-2 are the mean plasma concentration-time profiles for [5(4,6-dimethyl-pyrimidin-2-yl)-hexahydro-pyrrolo[3,4-c]pyrrol-2-yl]-(2-fluoro-2-[1,2,3]triazol-2-yl-phenyl)-methanone (Compound A) formulations.

FIGS. 3-8 are the composite plasma concentration-time profiles for Compound A formulations.

FIGS. 9-11 are the individual and mean plasma pharmacokinetic parameters versus treatment plots for Compound A formulations.

FIG. 12 is a line graph of the time (min) from lights out to 10 minutes of sleep vs. the change from baseline at day 10/11.

FIG. 13 is a line graph of the time (min) of total sleep vs. the change from baseline at day 10/11.

FIG. 14 is a line graph of the latency to persistent sleep (LPS) change in Hamilton Depression Rating Scale (HAM-D6) score from baseline at day 10/11 vs. the HAM-D6 change from baseline at day 11.

FIG. 15 is a line graph of the total sleep time (TST) change in HAM-D6 score from baseline at day 10/11 vs. the HAM-D6 score change from baseline at day 11.

FIG. 16 is the process flow chart regarding the preparation of the tablets used herein.

### DETAILED DESCRIPTION OF ILLUSTRATIVE EMBODIMENTS

### I. Definitions

The term “depression” includes major depressive disorder, persistent depressive disorder, depression associated with bipolar disorder (aka bipolar depression), seasonal affective disorder, psychotic depression, postpartum depression, premenstrual dysphoric disorder, situational depression, anhedonia, melancholy, mid-life depression, late-life depression, depression due to identifiable stressors, treatment resistant depression, or combinations thereof. In certain embodiments, the depression is major depressive disorder. In other embodiments, the major depressive disorder is with melancholic features or anxious distress.

The methods described herein are useful in the treatment of the core (or psychic) symptoms of depression. These symptoms include depressed mood and loss of interest or pleasure in nearly all activities.

The term “sleep onset” refers to the transition from wakefulness into non-rapid eye movement (NREM) sleep; and “sleep” generally refers to non-rapid eye movement (NREM) or rapid eye movement (REM) sleep.

The term “awake” describes a reasonably alert state of consciousness characterized by alpha and beta waves as detected by electroencephalogram, voluntary rapid eye movements and/or eye blinks. In other embodiments, an awake state may be characterized as the absence of NREM or REM sleep.

The term “night” includes the period of time from sunset to sunrise, occurring once each twenty-four hours. In some embodiments, night refers to a timeframe in a twenty-four period in a day that precedes sleep by a subject.

An “insomnia disorder” refers to a diagnosis using criteria found in the American Psychiatric Association's fifth edition of the Diagnostic and Statistical Manual of Mental Disorders (DSM-V) and the Third Edition of the World Health Organization's International Classification of Sleep Disorders (ICSD-3). In some embodiments, an “insomnia disorder” includes the difficulty initiating or maintaining sleep and waking too early and/or obtaining non-restorative sleep, where the sleep difficulty results in some form of daytime impairment.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the terms “treating”, “treatment” and the like, shall include the management and care of a subject or patient (preferably mammal, more preferably human) for the purpose of combating a disease, condition, or disorder and include the administration of a compound described herein to prevent the onset of the symptoms or complications, alleviate the symptoms or complications, or eliminate the disease, condition, or disorder. Similarly, “treatment” is used to encompass (a) reduction in the frequency of one or more symptoms; (b) reduction in the severity of one or more symptoms; (c) the delay or avoidance of the development of additional symptoms; and/or (d) delay or avoidance of the development of the disorder or condition, or any combination thereof.

As used herein, unless otherwise noted, the terms “subject” and “patient” may be used interchangeably and refer to an animal, preferably a mammal, most preferably a human, who has been the object of treatment, observation or experiment. In some embodiments, the subject or patient has experienced and/or exhibited at least one symptom of the disease or disorder to be treated and/or prevented. One skilled in the art will further recognize that the methods of treatment are directed to subjects or patients in need of such treatment, prevention or dosing regimen, more particularly to subjects or patients diagnosed with or exhibiting at least one symptom of depression (preferably, meeting the criteria for major depressive disorder or episode) regardless of type or underlying cause. In further embodiments, the subject is not suffering from or diagnosed with an insomnia disorder.

One skilled in the art will recognize that wherein methods of prevention are described, a subject in need thereof (i.e. a subject in need of prevention) shall include any subject who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of having new episodes of depression (and therefore in need of secondary prevention or preventive treatment) as a consequence of the subject's medical history, including, but not limited to, family history, pre-disposition, co-existing (comorbid) disorders or conditions, genetic testing, and the like.

Further, some of the quantitative expressions herein are recited as a range from about value X to about value Y. It is understood that wherein a range is recited, the range is not limited to the recited upper and lower bounds, but rather includes the full range from about value X through about value Y, or any value or range of values therein.

As used herein, the terms “including”, “containing” and “comprising” are used herein in their open, non-limiting sense.

### II. The Compounds

As discussed above, the compounds described herein are orexin-2 antagonists and may be used in the treatment of depression. In some embodiments, the compounds are administered such that they have a time to maximal plasma concentration of less than about 3 hours, less than about 2 hours, and preferably less than about 1 hour, i.e., less than about 45 minutes, less than about 30 minutes, less than about 15 minutes, among others. In other embodiments, the compound has an elimination half-life of about 4 hours and typically less than about 4 hours. For example, certain compounds of the present disclosure have a half-life of about 2 to about 3 hours, e.g., about 2 hours, about 2.1 hours, about 2.2 hours, about 2.3 hours, about 2.4 hours, about 2.5 hours, about 2.6 hours, about 2.7 hours, about 2.8 hours, or about 2.9 hours to about 3 hours. Given the short half-life, the amount of the compound remaining in the subject upon waking is typically below the threshold required for pharmacodynamic effect. For example, the compounds of the present disclosure typically have a pharmacodynamic effect from a dose level greater than about 5 mg.

In certain embodiments, the compound has the structure of formula (I):

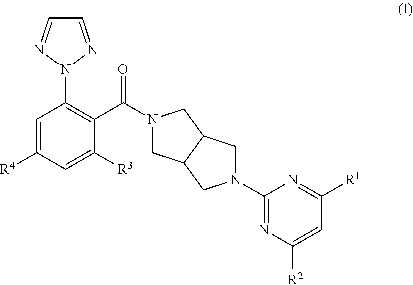

R¹ is C₁₋₄ alkyl. In some embodiments, R¹ is CH₃.

R² is C₁₋₄ alkyl. In some embodiments, R² is CH₃.

R³ is H or halogen. In some embodiments, R³ is halogen. In other embodiments, R³ is fluorine. In further embodiments, R³ is H.

R⁴ is H or C₁₋₄ alkoxy. In some embodiments, R⁴ is H. In further embodiments, R⁴ is C₁₋₄alkoxy. In other embodiments, R⁴ is methoxy.

“Alkyl” refers to a straight- or branched-chain alkyl group having from 1 to 12 carbon atoms in the chain. Examples of alkyl groups include methyl, ethyl, n-propyl, isopropyl, butyl, isobutyl, sec-butyl, tert-butyl and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 5 to 7 ring atoms per carbocycle. Illustrative examples of cycloalkyl groups include the following entities, in the form of properly bonded moieties:

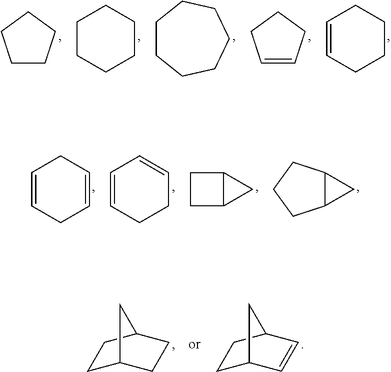

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

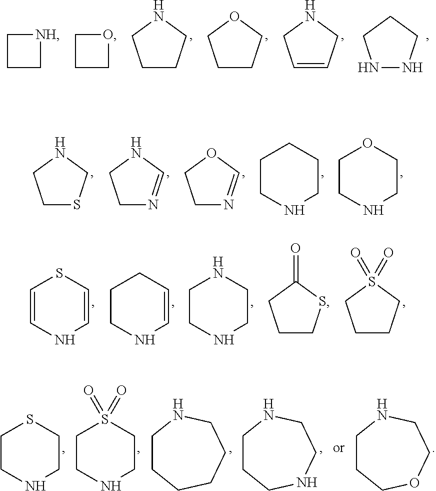

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

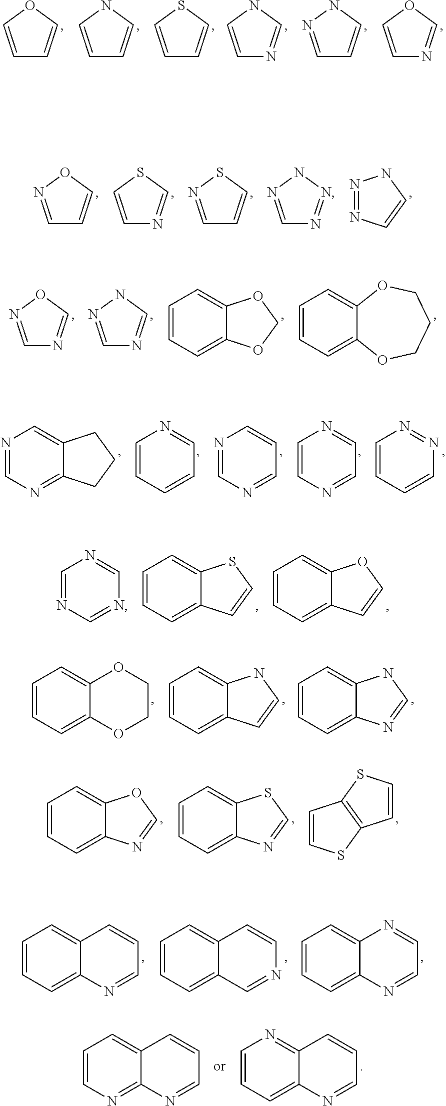

Those skilled in the art will recognize that the species of heteroaryl, cycloalkyl, and heterocycloalkyl groups listed or illustrated above are not exhaustive, and that additional species within the scope of these defined terms may also be selected.

“Alkoxy” includes a straight chain or branched alkyl group with a terminal oxygen linking the alkyl group to the rest of the molecule. Alkoxy includes methoxy, ethoxy, propoxy, isopropoxy, butoxy, t-butoxy, and so on.

“Halogen” represents chlorine, fluorine, bromine or iodine.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

The nomenclature “Cᵢ₋ⱼ” with j>i, when applied herein to a class of substituents, is meant to refer to embodiments for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≤N≤m, with m>n.

Any formula given herein is intended to represent a compound having a structure depicted by the structural formula as well as certain variations or forms. In particular, a compound of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

The compounds may include those described in U.S. Pat. No. 8,653,263 and US Patent Publication No. 2014/0171430, both of which are incorporated herein by reference. In some embodiments, the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof. In other embodiments, the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone. In further embodiments, the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone hydrochloride. In yet other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof. In still further embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl) methanone. In certain embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrate. In other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrochloride hydrate. In further embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl) phenyl) methanone hydrobromide hydrate.

Additionally, any formula given herein is intended to refer also to hydrates, solvates, and polymorphs of such compound, and mixtures thereof, even if such forms are not listed explicitly. A compound of Formula (I) or pharmaceutically acceptable salts of a compound of Formula (I) may be obtained as solvates. Solvates include those formed from the interaction or complexation of a compound with one or more solvents, either in solution or as a solid or crystalline form. In some embodiments, the solvent is water and then the solvates are hydrates. In addition, crystalline forms of a compound of Formula (I) or pharmaceutically acceptable salts of a compound of Formula (I) may be obtained as co-crystals. In certain embodiments, a compound of Formula (I) is obtained in a crystalline form. In other embodiments, a crystalline form of a compound of Formula (I) is cubic in nature. In other embodiments, pharmaceutically acceptable salts of compounds of Formula (I) are obtained in a crystalline form. In still other embodiments, compounds of Formula (I) are obtained in one of several polymorphic forms, as a mixture of crystalline forms, as a polymorphic form, or as an amorphous form. In other embodiments, a compound of Formula (I) converts in solution between one or more crystalline forms and/or polymorphic forms.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorus, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with 14° C.), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or an I¹²³ for SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

Also included are pharmaceutically acceptable salt of a compound of Formula (I) and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, G. S. Paulekuhn, “Trends in Active Pharmaceutical Ingredient Salt Selection based on Analysis of the Orange Book Database”, J. Med. Chem., 2007, 50:6665-72, S. M. Berge, “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-1-sulfonates, naphthalene-2-sulfonates, and mandelates.

The desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid, or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

When the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary, or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-D-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

Pharmaceutically acceptable prodrugs of a compound of Formula (I) and treatment methods employing such pharmaceutically acceptable prodrugs are also contemplated. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Exemplary prodrugs include compounds having an amino acid residue, or a polypeptide chain of two or more (e.g., two, three, or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl) amines. Examples of esters include C₁₋₇alkyl, C₅₋₇cycloalkyl, phenyl, and phenyl (C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher, Adv. Drug Delivery Rev. 1996, 19, 115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Robinson, J. Med. Chem. 1996, 39(I),10-18. Free amines can also be derivatized as amides, sulfonamides, or phosphonamides. All of these prodrug moieties may incorporate groups including ether (—O—), amine (—N—), and carboxylic acid (COO—) functionalities.

### III. Compositions

The compounds described herein, including the compounds of formula (I), may be formulated as a pharmaceutical composition to administration to a subject. Accordingly, a pharmaceutical composition may comprise (a) an effective amount of at least one compound described herein and (b) a pharmaceutically acceptable excipient. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds described herein may be prepared using suitable pharmaceutical excipients and compounding techniques known or that become available to those skilled in the art. The compositions may be administered in the inventive methods by a suitable route of delivery, e.g., oral, parenteral, rectal, topical, ocular routes, or by inhalation.

The preparation may be in the form of tablets, capsules, sachets, dragees, powders, granules, lozenges, powders for reconstitution, or liquid preparations. In some embodiments, the compositions are formulated for intravenous infusion, topical administration, or oral administration. In certain embodiments, the compositions are formulated for immediate release.

For oral administration, the compounds can be provided in the form of tablets or capsules, or as a solution, emulsion, or suspension. In certain embodiments, the compounds may be taken with food.

Oral tablets may include a compound mixed with pharmaceutically acceptable excipients such as inert fillers, diluents, disintegrating agents, binding agents, lubricating agents, sweetening agents, flavoring agents, coloring agents, glidants and preservative agents. Suitable inert fillers include sodium and calcium carbonate, sodium and calcium phosphate, lactose, lactose monohydrate, starch, sugar, glucose, methyl cellulose, magnesium stearate, mannitol, sorbitol, hypromellose, and the like. Exemplary liquid oral excipients include ethanol, glycerol, water, and the like. Starch, polyvinyl-pyrrolidone, sodium starch glycolate, microcrystalline cellulose, crospovidone (cross-linked polyvinyl N-pyrrolidone or PVP), and alginic acid are suitable disintegrating agents. Binding agents may include hypromellose (hydroxypropyl methylcellulose or HPMC), starch and gelatin. The lubricating agent, if present, may be magnesium stearate, stearic acid, or talc. The glidant, if present, may be silica (SiO₂) such as colloidal silica. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, the compound may be mixed with a solid, semi-solid, or liquid diluent. Soft gelatin capsules may be prepared by mixing the compound with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups or may be lyophilized or presented as a dry product for reconstitution with water or other suitable vehicle before use. Such liquid compositions may optionally contain pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds described herein may also be administered by non-oral routes. For example, the compounds may be formulated for rectal administration. For parenteral use, including intravenous, intramuscular, or intraperitoneal routes, the compound may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of the compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compound may utilize a patch formulation to affect transdermal delivery.

Compounds may alternatively be administered by inhalation, via the nasal or oral routes, e.g., in a spray formulation also containing a suitable carrier.

### IV. Methods of Treating Depression

As described herein, the inventors found a surprising, robust antidepressant effect when using the compounds described on subjects diagnosed with depression. Although not intending to be limited by theory, it is believed that because the activity of orexin containing neurons is negligible during sleep (typically at night), the antidepressant efficacy of the compounds discussed herein is surprising. As disclosed herein, administration prior to sleep (typically at night) of the compounds of the disclosure is associated with statistically significant antidepressant efficacy, with the efficacy not related to the effect on sleep items.

Accordingly, methods of treating a subject suffering from or diagnosed with depression are provided. These methods comprise administering to a subject in need of such treatment an effective amount of a compound described herein. In certain embodiments, the compound is of formula (I).

The compound is preferably administered once daily and is administered to the subject prior to sleep. For example, the compound is administered within about 2 hours of sleep, within about 1 hour, or within about 30 minutes before sleep. In other embodiments, the compound is administered at least about 4 hours before the subject wakes or intends to wake from sleep, including about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, or about 12 hours before the subject wakes or intends to wake from sleep. In certain embodiments, the compound is administered at least 6 hours to about 12 hours before the subject wakes or intends to wake from sleep. In preferred embodiments, the compound is administered at night.

After administration of the compound, the compound undergoes at least one half-life before the subject wakes from sleep. In other embodiments, the compound undergoes at least two half-lives, and preferably at least three half-lives before the subject wakes from sleep.

Desirably, the compound is below the threshold required for pharmacodynamic effect after about 6 to about 8 hours after administration of the compound. This differs from antidepressants in the art which are designed to achieve a steady state concentration of the antidepressant in the patient. The methods described herein differ in that after one to eight hours of administration of the drug, the concentration of the drug will fall below pharmacodynamic levels and remain at those levels for the remainder of the 24-hour treatment period until the next dose of drug is administered.

Therapeutically effective amounts for the compounds described herein include amounts that elicit the biological or medicinal response in a tissue system, animal or human that is being sought by a researcher, veterinarian, medical doctor, or other clinician, which includes alleviation of the symptoms of the disease or disorder being treated. Optimal dosages to be administered may be readily determined by those skilled in the art, and may vary with the mode of administration, the strength of the preparation and the advancement of the disease condition. Such factors including the particular patient being treated, including patient's sex, age, weight, diet, time of administration and concomitant diseases, among others. In certain embodiments, the effective amount of each dose of the compounds described herein is about 0.001 to about 200 mg of compound per kg of subject's body weight per day, about 0.05 to 100 mg/kg/day, or about 1 to 35 mg/kg/day, in single or divided dosage units (examples of such dosage units include 2.5 mg, 5 mg, 10 mg, and 20 mg tablets). For a 70-kg human, an illustrative range for a suitable dosage amount is from about 0.05 to about 7 g/day, or about 0.2 to about 2.5 g/day.

The effective amount of the compound described herein may also be described without reference to the weight of the subject. Accordingly, the effective amount of the compound is about 10 to about 60 mg. In some embodiments, the effective amount of the compound is about 10 mg, about 15 mg, about 20 mg, about 25 mg, about 30 mg, about 35 mg, or about 40 mg, or within a range defined by any two of these values.

The effective amount of the compound may be administered in a single daily dose. In further embodiments, the compound is administered daily and one or more symptoms of the depression is reduced or ameliorated within about 11 days of a first administration, i.e., day 1.

Frequency adjustment can be accomplished by a one-time switch in frequency or may be determined over two or more administrations. By doing so, the attending physician or the like may determine an optimal frequency for administration and thereby tailor the administration to the patient.

Also contemplated by these methods is the administration of rescue doses of the compounds described herein. The term “rescue dose” as used herein refers to one or more additional doses of a compound described herein in addition to the regularly prescribed dose. The amount of a compound described herein in the rescue dose may be determined by the prescribing physician or clinician and will depend on any of the factors discussed herein. In certain embodiments, the rescue dose of a compounds described herein is the same as the effective dose used during the normal administration schedule. In other embodiments, the rescue dose differs from the effective dose used during the normal administration schedule.

One skilled in the art will recognize that in the methods described herein, the maintenance of the response in a patient may be determined by for example, a clinician, physician, psychiatrist, psychologist, or other suitable medical professional. Additionally, maintenance of the antidepressant response may be established by for example, an absence of relapse of the depression (or one or more symptoms of the depression), an absence of the need for additional or alternate treatment(s) for the depression, or an absence of the worsening of the depression. The physician or attending clinician may utilize any technique known in the art including, without limitation, general patient evaluation, diagnostic questionnaires, and evaluations such as the Clinical Global Impression-Severity (CGI-S) scale, EuroQol; 5 dimension; 5 level (EQ-5D-5L), Patient Health Questionnaire-9 Item (PHQ-9), Sheehan Disability Scale (SDS), Inventory of Depressive Symptomatology-Clinician rated, 30-item scale (IDS-C30), Montgomery-Åsberg Depression Rating Scale (MADRS) questionnaire, Hamilton rating scale for depression (HAM-D or HDRS) Beck Scale for Depression, or Quick Inventory of Depressive Symptomology (QIDS). The frequency may be evaluated and/or changed if the score from one or more of the above-noted scales or questionnaire changes.

In addition, the compounds may be used in combination with additional active ingredients in the treatment of the above conditions. The additional active ingredients may be administered simultaneously, separately or sequentially. In some embodiments, the additional active ingredients are effective in the treatment of conditions, disorders, or diseases mediated by orexin activity, such as another orexin modulator or a compound active against another target associated with the particular condition, disorder, or disease. The combination may serve to increase efficacy (e.g., by including in the combination a compound potentiating the potency or effectiveness of a compound herein), decrease one or more side effects, or decrease the required dose of the compound described herein or additional active agent. In certain embodiments, the additional active ingredient is an antidepressant. In other embodiments, the additional active ingredient is a monoaminergic antidepressant.

Accordingly, the compound of formula (I) may be used in combination with a second antidepressant. The second antidepressant may be a conventional drug used to combat depression such as N-methyl-D-aspartate receptor antagonists, norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), noradrenergic and specific serotonergic antidepressants (NaSSAs), corticotropin releasing factor (CRF) antagonists, alpha-adrenoreceptor antagonists and atypical antidepressants. In some embodiments, the N-methyl-D-aspartate (NMDA) receptor antagonist is ketamine including its racemates esketamine, arketamine, or combinations thereof. In further embodiments, the norepinephrine reuptake inhibitor includes amitriptyline, clomipramine, doxepin, imipramine, trimipramine, amoxapine, desipramine, maprotiline, nortriptyline, protriptyline, reboxetine, or pharmaceutically acceptable salts thereof. In other embodiments, the selective serotonin reuptake inhibitor includes fluoxetine, fluvoxamine, paroxetine, sertraline, or pharmaceutically acceptable salts thereof. In further embodiments, the monoamine oxidase inhibitor includes isocarboxazid, phenelzine, tranylcypromine, selegiline and pharmaceutically acceptable salts thereof. In yet other embodiments, the reversible inhibitor of monoamine oxidase includes moclobemide or pharmaceutically acceptable salts thereof. In still further embodiments, the serotonin and noradrenaline reuptake inhibitor includes venlafaxine or pharmaceutically acceptable salts thereof. In other embodiments, the atypical antidepressant includes bupropion, lithium, nefazodone, trazodone, viloxazine, sibutramine, or pharmaceutically acceptable salts thereof. In yet further embodiments, the second antidepressant includes adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine zometapine, or pharmaceutically acceptable salts thereof; or St. John's wort herb, Hypericum perforatum, or extracts thereof.

In some embodiments, the compound of formula (I) is co-administered with esketamine. In further embodiments, the compound of formula (I) is administered separately from esketamine such as, e.g., sequentially. The compound of formula (I) may be administered prior or subsequent to esketamine.

### V. Kits

Also described herein are kits for administering one or more compounds described herein to a patient for the treatment of depression. The representative kits include one or more dosage units comprising an effective amount of one or more compounds described herein for administration to a patient and at a given frequency.

The dosage unit may be formulated for delivery by any means. In certain embodiments, the dosage unit is formulated for oral, intravenous, intranasal, intramuscular, sublingual, transdermal, otic, or rectal delivery. In certain embodiments, the dosage unit is formulated for oral delivery.

The dosage unit may be formulated to contain any amount of a compound described herein, depending on the route of administration. Accordingly, each dosage unit may comprise the required dosage for the patient or may comprise a portion of a compound described herein which is required for a single dosage.

Also optionally included in the kits is a depression symptom rating scale questionnaire. The questionnaire may be for use by the patient alone or in combination with a physician. The questionnaire may be useful for determining the level of depression of the patient at any stage of compound administration. In one embodiment, the questionnaire is one or more of the questionnaires noted herein.

Instructions for performing the claimed methods and administering the compound may also be included in the kits described herein.

The kits may be organized to indicate a single formulation containing a compound described herein or combination of formulations, each containing a compound described herein. The composition may be sub-divided to contain appropriate quantities of a compound described herein. The unit dosage can be packaged compositions such as packeted powders, vials, ampoules, prefilled syringes, tablets, caplets, capsules, or sachets containing liquids.

The compound described herein may be a single dose or for continuous or periodic discontinuous administration. For continuous administration, a kit may include a compound described herein in each dosage unit. When varying concentrations of a compound described herein, the components of the composition containing the compound described herein, or relative ratios of the compound described herein or other agents within a composition over time is desired, a kit may contain a sequence of dosage units.

The kit may contain packaging or a container with a compound described herein formulated for the desired delivery route. The kit may also contain dosing instructions, an insert regarding the compound described herein, instructions for monitoring circulating levels of the compound, or combinations thereof. Materials for using the compound may further be included and include, without limitation, reagents, well plates, containers, markers, or labels, and the like. Such kits may be packaged in a manner suitable for treatment of a desired indication

Other suitable components to include in such kits will be readily apparent to one of skill in the art, taking into consideration the desired indication and the delivery route. The kits also may include, or be packaged with, instruments for assisting with the injection/administration of the compound to the patient. Such instruments include, without limitation, an inhalant, syringe, pipette, forceps, measuring spoon, eye dropper, or any such medically approved delivery means. Other instrumentation may include a device that permits reading or monitoring reactions in vitro.

The compound may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In certain embodiments, the package is a labeled blister package, dial dispenser package, or bottle.

Methods for optimizing a dosage of the compound for a patient having or being predisposed to depression also are provided. These methods can include (a) administering an effective amount of the compound to the patient, (b) analyzing the effects of the compound, and (c) administering an effective amount of the compound to the patient less frequently of a defined duration.

### VI. Aspects

The present disclosure comprises at least the following aspects.

Aspect 1. A method of treating a subject suffering from or diagnosed with depression, comprising administering to a subject in need of such treatment an effective amount of a compound of formula (I):

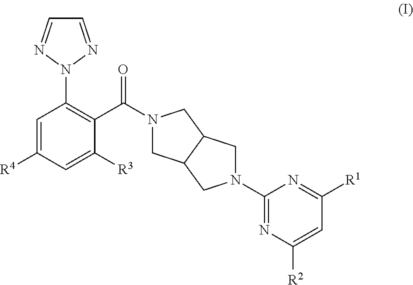

wherein, R¹ is C₁₋₄ alkyl; R² is C₁₋₄ alkyl; R³ is H or halogen; and R⁴ is H or C₁₋₄ alkoxy; or a pharmaceutically acceptable salt or hydrate thereof, wherein the compound is administered prior to sleep.

Aspect 2. The method of aspect 1 wherein the subject is not suffering from or diagnosed with an insomnia disorder.

Aspect 3. The method of any one of the preceding aspects, wherein R³ is halogen.

Aspect 4. The method of any one of the preceding aspects, wherein R³ is fluorine.

Aspect 5. The method of any one of the preceding aspects, wherein R⁴ is H.

Aspect 6. The method of aspect 1 or 2, wherein R⁴ is C₁₋₄alkoxy.

Aspect 7. The method of any one of aspects 1, 2, or 6, wherein R⁴ is methoxy.

Aspect 8. The method of any one of aspects 1, 2, 6, or 7, wherein R³ is H.

Aspect 9. The method of any one of the preceding aspects, wherein R¹ is CH₃.

Aspect 10. The method of any one of the preceding aspects, wherein R² is CH₃.

Aspect 11. The method of aspect 1 or 2, wherein the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof.

Aspect 12. The method of aspect 1, 2, or 11, wherein the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone.

Aspect 13. The method of aspect 1, 2, or 11, wherein the compound is 5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone hydrochloride.

Aspect 14. The method of aspect 1 or 2, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof.

Aspect 15. The method of any one of aspects 1, 2, or 12, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone

Aspect 16. The method of any one of aspects 1 to 3, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrate.

Aspect 17. The method of any one of aspects 1 to 3, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrochloride hydrate.

Aspect 18. The method of any one of aspects 1 to 3, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrobromide hydrate.

Aspect 19. The method of any one of the preceding aspects, wherein the compound is administered at night.

Aspect 20. The method of any one of the preceding aspects, wherein the compound is administered such that it has a time to maximal plasma concentration of less than about 1 hour.

Aspect 21. The method of any one of the preceding aspects, wherein the compound has an elimination half-life of about 2 to about 3 hours.

Aspect 22. The method of any of the preceding aspects, wherein the compound is administered to the subject about at least 4 hours before the subject intends to wake from sleep.

Aspect 23. The method of any one of the preceding aspects, wherein the compound is below the threshold required for pharmacodynamic effect after about 6 to about 8 hours after administration of the compound.

Aspect 24. The method of any one of the preceding aspects, wherein the compound undergoes at least two half-lives before the subject wakes from sleep.

Aspect 25. The method of any one of the preceding aspects, wherein steady state of the compound is not achieved.

Aspect 26. The method of any one of the preceding aspects, wherein the compound is administered daily.

Aspect 27. The method of any one of the preceding aspects, wherein the compound is administered orally.

Aspect 28. The method of any one of the preceding aspects, wherein the effective amount is about 0.05 to about 100 mg/kg/day.

Aspect 29. The method of any one of the preceding aspects, wherein the effective amount is about 10 to about 40 mg.

Aspect 30. The method of any one of the preceding aspects, wherein the compound is administered daily and one or more symptoms of the depression is reduced or ameliorated within about 11 days of a first administration.

Aspect 31. The method of any one of the preceding aspects, wherein the depression comprises major depressive disorder, persistent depressive disorder, depression associated with bipolar disease, seasonal affective disorder, psychotic depression, postpartum depression, premenstrual dysphoric disorder, situational depression, anhedonia, melancholy, mid-life depression, late-life depression, depression due to identifiable stressors, treatment resistant depression, or combinations thereof.

Aspect 32. The method of aspect 31, wherein the depression is major depressive disorder.

Aspect 33. The method of aspect 32, wherein the major depressive disorder is with melancholic features or anxious distress.

Aspect 34. The method of any one of the preceding aspects, further comprising administering a second antidepressant.

Aspect 35. The method of aspect 34, wherein said second antidepressant is a norepinephrine reuptake inhibitor, selective serotonin reuptake inhibitor, monoamine oxidase inhibitor, reversible inhibitor of monoamine oxidase, serotonin and noradrenaline reuptake inhibitor, noradrenergic and specific serotonergic antidepressant, corticotropin releasing factor antagonist, alpha-adrenoreceptor antagonists, atypical antidepressant, NMDA antagonist, or combinations thereof.

Aspect 36. The method of aspect 35, wherein said NMDA antagonist is esketamine.

The following Examples are set forth to aid in the understanding of the disclosure, and are not intended and should not be construed to limit in any way the invention set forth in the claims which follow thereafter.

### EXAMPLES

### Example 1

This example was performed to determine the plasma pharmacokinetic (PK) and bioavailability of a solid dose formulation of [5 (4,6-dimethyl-pyrimidin-2-yl)-hexahydro-pyrrolo[3,4-c]pyrrol-2-yl]-(2-fluoro-2-[1,2,3]triazol-2-yl-phenyl)-methanone (Compound A) after single dose tablet administration relative to a suspension formulation. Also addressed are the effect of a semi-fasted condition on the rate and extent of bioavailability of the solid dose formulation and tolerability of the solid and oral suspension formulations.

### (i) Reagents and Testing Parameters

[5(4,6-Dimethyl-pyrimidin-2-yl)-hexahydro-pyrrolo[3,4-c]pyrrol-2-yl]-(2-fluoro-2-[1,2,3]triazol-2-yl-phenyl)-methanone (Compound A) was prepared as described in method B in Example 107 U.S. Pat. No. 8,653,263 with the exception that the recrystallization was performed using ethanol instead of an ethanol/2-propanol mixture.

The internal standard was isotope labeled [5 (4,6-dimethyl-pyrimidin-2-yl)-hexahydro-pyrrolo[3,4-c]pyrrol-2-yl]-(2-fluoro-2-[1,2,3]triazol-2-yl-phenyl)-methanone which has the following structure.

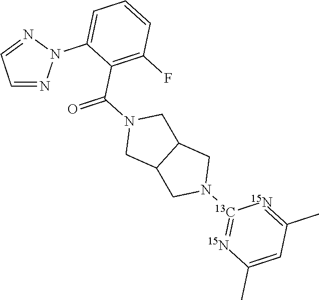 [SMILES: Cc1cc(C)[15n][13c](N2CC3CN(C(=O)c4c(F)cccc4-n4nccn4)CC3C2)[15n]1]

The internal standard was prepared described in method B in Example 107 U.S. Pat. No. 8,653,263 with the exception that step b was performed using isotope labeled Intermediate 92, i.e., 2-(4,6-dimethylpyrimidin-2-yl)octahydropyrrolo[3,4-c]pyrrole, bis-HCl salt, which was prepared using isotope labelled 2-chloro-4,6-dimethyl pyrimidine of the following structure:

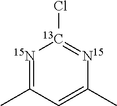

During each treatment period, blood samples were collected for PK measurements. Specifically, venous blood samples of 3 mL each were collected for determination of Compound plasma concentrations. The following plasma Compound A PK parameters were estimated using the actual times of blood sampling:
Cₘₐₓ peak plasma concentration.
tₘₐₓ time to reach the peak plasma concentration.
AUCₗₐₛₜ area under the plasma concentration-time curve from 0 to t hours post study drug dosing, calculated by trapezoidal summation (time t is the time of the last quantifiable concentration Cₗₐₛₜ).
AUC_{∞}AUCₗₐₛₜ extrapolated to infinity, calculated as AUCₗₐₛₜ+Cₗₐₛₜ/γ_{z}.
γ_{z} elimination rate constant, determined by linear regression of the terminal points of the In-linear plasma concentration-time curve.
t_{1/2} terminal half-life, defined as 0.693/γ_{z}.
CL/F total clearance of drug after extravascular administration, uncorrected for absolute bioavailability, calculated as Dose/AUC∞.
Vd/F apparent volume of distribution after extravascular administration, uncorrected for absolute bioavailability.

Compound A plasma levels were determined using LC-MS/MS using the equipment and parameters set forth in Tables 1-4.

TABLE 1
HPLC System

| | LC-10 Advp (Shimadzu) with |
|---|---|
| | SCL-10 Avp system controller |
| Pump | and DGI-14A on-line degasser |
| Pressure limits (psi) | 0-3500 |
| Pumping Mode | Binary |
| Total Flow (mL/min) | 0.350 |
| Initial pump B conc. (%) | 20.0 |
| Column heater | Shimadzu CTO10ACvp |
| Oven temperature (° C.) | 30.0 |
| Autosampler | SIL HTc (Shimadzu) |
| Injection Volume (μL) | 2.00 |
| Cooler Temperature (° C.) | 4 |

Shimadzu SIL-HTc Properties

| | |
|---|---|
| Rinsing Volume (μL) | 500 |
| Needle Stroke (mm) | 47 |
| Rinsing Speed (μL/s) | 35 |
| Sampling Speed (μL/s) | 5.0 |
| Purge Time (min) | 1.0 |
| Rinse Dip Time (sec) | 5 |
| Rinse mode | Before and after aspiration |

Gradient

| Time (min) | Module | Events | Parameter |
|---|---|---|---|
| 1.50 | Pumps | Pump B Conc. | 50 |
| 1.51 | Pumps | Pump B Conc. | 90 |
| 2.50 | Pumps | Pump B Conc. | 90 |
| 2.51 | Pumps | Pump B Conc. | 20 |
| 3.00 | Controller | Stop | |

TABLE 2
Detector

| | |
|---|---|
| Detector | Mass spectrometer API |
| | 4000 (AB Sciex) |
| Ion Source | Turbo-ion spray |
| Duration (min) | 4.00 |
| Polarity | Positive MRM |
| Resolution Q1 | unit |
| Resolution Q3 | unit |
| Intensity threshold (cps) | 0.00 |
| CUR | 30.0 |
| CAD | 5.00 |
| GAS 1 (psi) | 40.0 |
| GAS 2 (psi) | 50.0 |
| IS (V) | 5000 |
| Temperature (° C.) | 600 |
| Ihe | On |

TABLE 3
Mass Dependent Parameters

| | Q1 | Q3 | DP | CE | CXP | Time |
|---|---|---|---|---|---|---|
| Compound | Mass | Mass | (V) | (V) | (V) | (ms) |
| Compound A | 408.2 | 190.0 | 80 | 39 | 13 | 300 |
| Internal | 411.2 | 190.0 | 80 | 39 | 13 | 300 |
| Standard | | | | | | |
| Compound B | | | | | | |

TABLE 4
Blank Matrix

| Matrix | Species | Anti-coagulant |
|---|---|---|
| Plasma | Human | EDTA |

| | |
|---|---|
| Analytical column | XBridge BEH C18 column |
| Dimensions (mm) | 50 × 2.1 |
| Particle Size (um) | 3.5 |
| Typical backpressure | 1500 |
| Pre-column/filter | Frit filter |

HPLC Reagents

| | |
|---|---|
| Mobile phase A | 0.1% formic acid in water |
| | Mix formic acid (2.00 mL) with |
| | water (2000 mL) |
| Mobile phase B | acetonitrile |
| Rinse Solvent | 2-propanol:acetonitrile:water:formic acid |
| | (40:40:20:0.1 vv/v/v/v) |
| | Mix propanol (400 mL) with acetonitrile |
| | (400 mL) with water (200 mL) and |
| | formic acid (1 mL) |
| Dilution Solvent | 0.1% formic acid in water |
| | Mix formic acid (1.00 mL) with |
| | water (1000 mL) |
| Stock Dilution | dimethylsulfoxide:acetonitrile (50:50, v/v) |
| Solvent | Mix dimethylsulfoxide (50.0 mL) with |
| | acetonitrile (50.0 mL) |
| System Check | 0.1% formic acid in water: |
| Dilution Solvent | acetonitrile (80:20, v/v) Mix 0.1% |
| | formic acid (80.0 mL) in water |
| | with acetonitrile (20.0 mL) |

Two stock solutions were prepared for Compound A and one stock solution for internal standard Compound B according to the following.
Compound A Stock Solution: this solution was prepared by dissolving Compound A (1.00 mg) in the Stock Dilution Solvent (10.0 mL)
Compound A Overcurve Stock Solution: this solution was prepared by dissolving Compound A (2.00 mg) in 2.00 mL of the Stock Dilution Solvent (2.00 mL)
Compound B Stock Solution: this solution was prepared by dissolving Compound B (1.00 mg) in 10.0 mL of the Stock Dilution Solvent (2.00 mL).

Standard stock solutions were prepared for Compound A and the internal reference according to the following.
Compound A standard solution 1 (10.0 μg/mL): Compound A Stock Solution (1000 μL) was combined with the Stock Dilution Solvent (10.0 mL).
Compound A standard solution 2 (1.00 μg/mL): Compound A Stock Solution (100 μL) was combined with the Stock Dilution Solvent (10.0 mL).
Compound A Standard Solution 3 (0.100 μg/mL): Compound A Stock Solution (10.0 μL) was combined with the Stock Dilution Solvent (10.0 mL).
Compound B Working Solution (200 mg/mL): Compound B Stock Solution (200 μL) was combined with the Stock Dilution Solvent (100 mL).

Samples were prepared for testing using the following protocol:
(i) Plasma samples at room temperature were homogenized.
(ii) The samples were centrifuged for 5 minutes at about 2500×g and 20° C.
(iii) The plasma sample (50.0 μL) was pipetted into a 1.2 mL round well collection plate.
(iv) Stock Dilution Solvent (50.0 μL) was added to the blanks and the internal standard working solution was added to all other tube. Tubes were then vortexed for 10 seconds.
(v) Acetonitrile (100 μL) was added to each tube and the tubes again vortex-mixed for 10 seconds.
(vi) Acetonitrile (250 μL) was further added to each tube and the tubes again vortex-mixed for 60 seconds.
(vii) The samples were centrifuged for 5 minutes at about 2500×g and 20° C.
(viii) The supernatant (50.0 μL) was transferred into a 1.2 mL round well collection plate using the liquidator.
(ix) Formic acid (0.1%; 400 μL) in water was added to each tube and the tubes vortex-mixed for 10 seconds.

Plots of the chromatograms and peak area integrations were carried out by Analyst (version 1.6.2, MDS Sciex, Concord, Canada). Calculations were done using Watson 7.3 bioanalytical LIMS (Thermo Fisher Scientific).

### (ii) Drug Compositions

The suspension containing Compound A was prepared by reconstituting a powder (100 mg Compound A) with hypromellose (5 mg/mL) solution to provide an oral 5 mg/mL oral suspension of Compound A. The hypromellose used for reconstitution is a 0.5% hypromellose solution in sterile water for injection.

The specific procedure for preparing the suspension follows:
(i) A powder containing Compound A is was added to a vial.
(ii) To achieve the desired concentration of the suspension, an appropriate amount of 0.5% HPMC solution was added to the vial.
(iii) A clean stir bar was added to the vial.
(iv) Since it was necessary to suspend the drug substance, the vial with spin bar was placed on a magnetic stir plate and the speed was adjusted to gently create a vortex in the liquid. Once a gentle vortex was achieved, the speed of the stir bar was increased for a rapid vortex at 2500 RPM (about 2400 to about 2600).
(v) The composition was mixed for a minimum of about 24 to about 36 hours.
(vi) After mixing, the suspension was ready for use and the required volume was withdrawn for dosing.

Tablets containing Compound A contained the components set forth in Table 5.

TABLE 5

| | Quantity/Unit Dose | |
|---|---|---|
| Component | (in mg/tablet) | (in % w/w) |
| Compound A | 20 | 13.3 |
| Lactose monohydrate | 80 | 53.3 |
| Microcrystalline cellulose | 40.5 | 27 |
| Crospovidone | 7.5 | 5 |
| Silica colloidal | 1 | 0.7 |
| Magnesium stearate | 1 | 0.7 |
| Total | 150 | 100 |

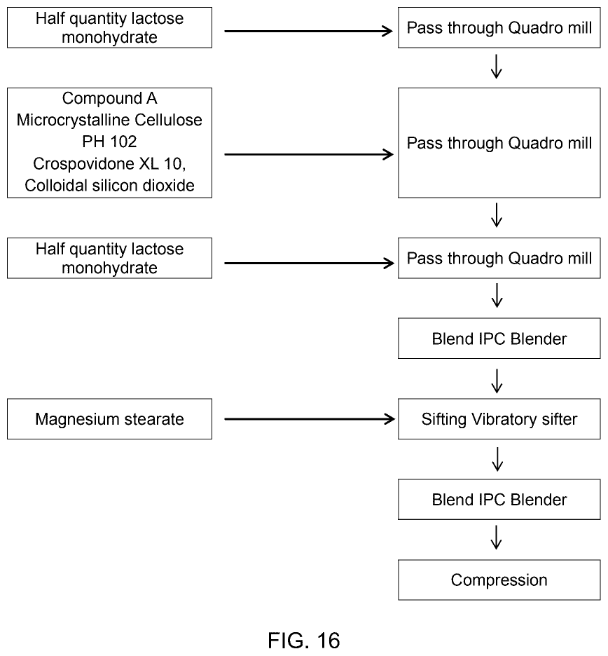

The tablets were prepared as described in FIG. 16 and according to the following direct compression process:
A. Screening & Blending
1. All materials were passed through a Quadro Comil using 07L039R03125 screen at 1000 rpm, with the exception of magnesium stearate. The following sequence of adding materials to the screen was followed:
   (i) About ½ of the lactose monohydrate
   (ii) Compound A
   (iii) Colloidal Silicon dioxide
   (iv) Crospovidone
   (v) Microcrystalline Cellulose
   (vi) About half of the lactose monohydrate
2. The mixture was blended for 20 minutes at 20 rpm.
3. Magnesium stearate was sifted through a #40 sieve.
4. The mixture of step 3 was blended for 5 minutes at 20 rpm.
B. Compression

The tablets were compressed using a rotary press fitted with a “D” type 7.0 mm round shallow concave punches and appropriate dies. The in-process parameters are set forth in Table 6.

TABLE 6

| Tablet Parameters | Limits |
|---|---|
| Description | White to off white circular |
| | biconvex tablets, plain on |
| | both sides |
| Friability | not more than (NMT) 1.0% |
| Hardness | 5.0-13.0 kp (target 8.0 kp) |
| Disintegration Time | NMT 15 minutes |
| Individual tablet weight | 150 mg ± 7.5% |
| variation | (139.0-161.0 mg) |
| Group Weight of | 1.5 g ± 5% (1.425 g-1.575 g) |
| 10 tablets | |
| Thickness | 3.35 mm-3.75 mm |
| | (target 3.50 mm) |

### (iii) Measurement of PK Parameters

Eighteen male subjects between 18 and 55 years of age, inclusive, were tested. The subjects had not received a potent cytochrome P450(CYP)3A and CYP2C19 inhibitor within 14 days or a period less than 5 times the drug's half-life (whichever was longer) or a potent CYP3A or CYP2C19 inducer within 30 days before study drug administration on Day 1 of Period 1 were excluded.

Subjects received a single oral dose of 20 mg Compound A (suspension or as a solid dose formulation) on Day 1 of each of the 3 treatment periods. The total study duration for each subject (including screening and follow-up visit) was to be about 7 to 8 weeks.

This study consisted of 3 phases: an eligibility screening examination (between 21 days and 2 days prior to first dose administration), a 3-way crossover single dose open-label treatment phase which consisted of 3 treatment periods separated by a washout period of at least 6 days between dosing, and a follow-up visit (within 7 to 14 days after last dose administration).

All subjects enrolled were randomly assigned to one of three Treatments:
Treatment A: 20 mg oral suspension formulation of Compound A (fasted state)
Treatment B: 20 mg solid formulation of Compound A (fasted state)
Treatment C: 20 mg solid formulation of Compound A (semi-fasted state)

### (iii) Results

A mixed-effect model was applied to the natural log transformed Cₘₐₓ and AUC. The model included sequence, period, treatment as fixed effects, and subject as a random effect. For each of the parameters, the comparisons included:
The solid dosage formulation (fasted) vs. the oral suspension formulation (fasted)
The solid dosage formulation (fasted) vs. the solid dosage formulation (semi-fasted)

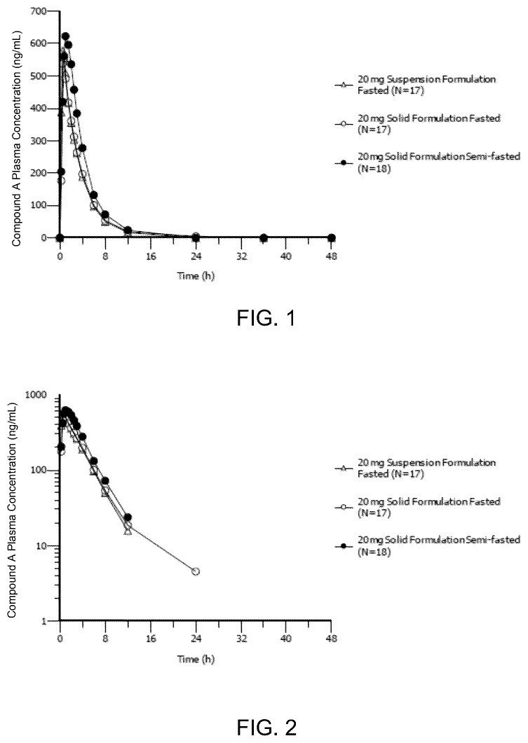

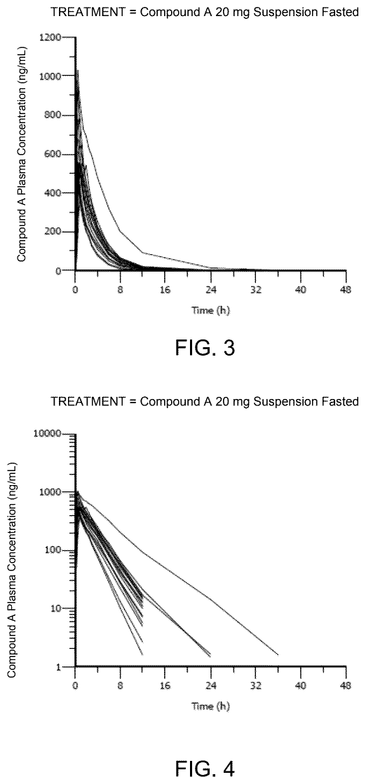

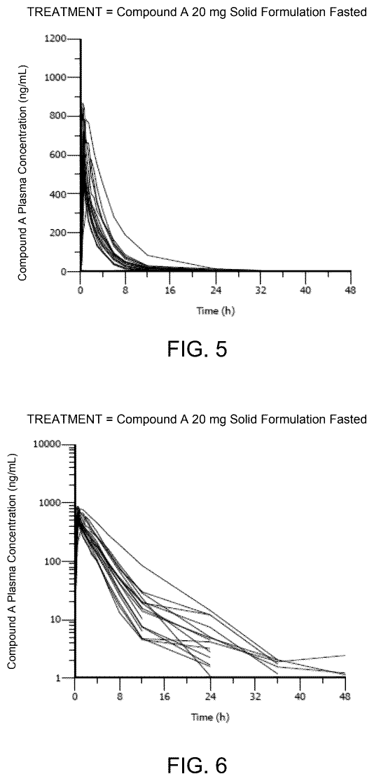

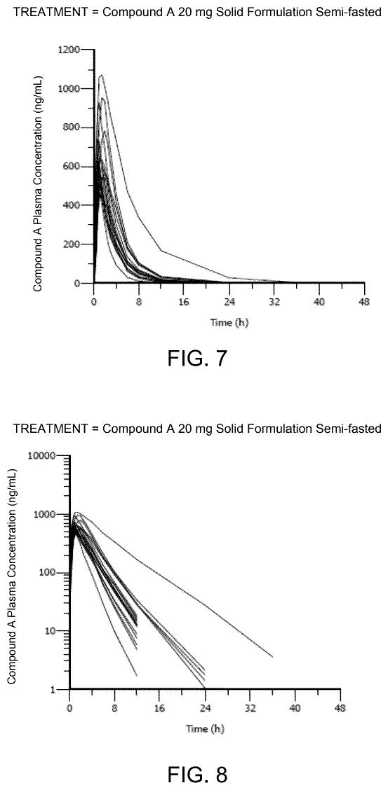

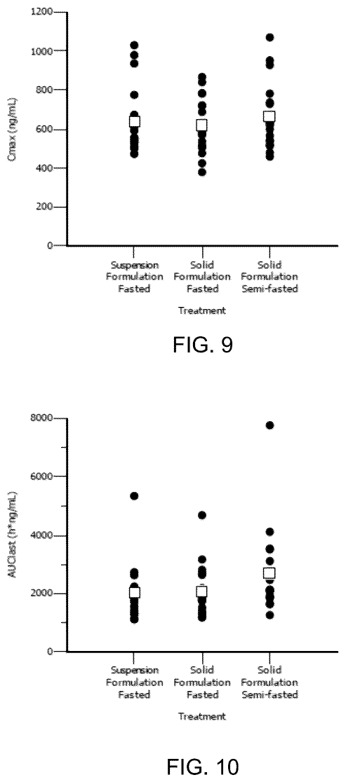

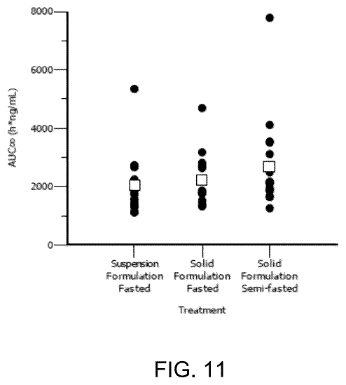

Following oral administration, Compound A was rapidly absorbed and reached Cₘₐₓ with median tₘₐₓ values ranging from 0.5 to 1.0 hour. Following Cₘₐₓ, Compound A concentrations declined rapidly in a mono-exponential manner (up to 12 hour postdose). Mean t_{1/2} values for the suspension (fasted) and tablet (semi-fasted) were similar (˜2 hours). However, mean t_{1/2} value for the tablet under fasting condition was longer than expected (˜5 hours). Extended low levels of plasma concentrations during the terminal phase were found in some subjects resulting in t_{1/2} values ranging from 1.9 to 17.3 hours. See, FIGS. 1-11.

### Example 2

This example was performed as a multi-center, double-blind, diphenhydramine and placebo-controlled study. Men and women with a diagnosis of MDD between the ages of 18 and 64, inclusive, were enrolled. At screening, the subjects had a total score of ≥30 on the IDS-C₃₀, corresponding to moderate to severe depression.

Blood and saliva were collected for the assessment of biomarkers, among others. Venous blood samples (3 mL each) were collected in fasting condition between 8:00 and 10:00 am for the determination of [5(4,6-dimethyl-pyrimidin-2-yl)-hexahydro-pyrrolo[3,4-c]pyrrol-2-yl]-(2-fluoro-2-[1,2,3] triazol-2-yl-phenyl)-methanone plasma concentrations and biomarkers related to immune system activity, Hypothalamus pituitary adrenal (HPA) axis activation, neurotropic factors and metabolic factors were measured. Pharmacokinetic (PK) blood samples also were collected. Plasma samples were analyzed to determine concentrations of Compound A using LC-MS/MS. Saliva was collected for the measurement of concentrations of cortisol. Saliva concentrations of cortisol were added as a biomarker.

Forty-eight subjects were randomly assigned (in a 2:1:1 ratio) to 20 mg of Compound A, 25 mg diphenhydramine or placebo q.d. (once daily) in the evening over 10 days for women of child bearing potential (WOCBP) or 4 weeks for males and women of non-child bearing potential (WONCBP). The subjects received the medication as capsules containing 1 tablet of 20 mg Compound A, 1 tablet of 25 mg diphenhydramine or placebo. Males and WONCBP took 1 capsule every evening just before bedtime from Day 1 to Day 28. WOCBP took 1 capsule every evening just before bedtime from Day 1 to Day 10. There were 2 follow up visits occurring at 3 and 14 days.

For the evaluation of symptoms of depression, assessments were done at screening and during the study. Specifically, symptoms of depression were performed using the Mini International Neuropsychiatric Interview (MINI) 6.0 interview, or Hamilton rating scale for Depression-17 (HDRS17). Also, polysomnography (PSG) was performed to quantify sleep stages including latency to persistent sleep (LSP) and total sleep time (TST). Thereafter, symptoms of depression were taking on days 11 and 29 during treatment and days 3 and 14 following treatment. PSG was recorded overnight after the first and tenth dose administration of study medication.

### (i) Posology

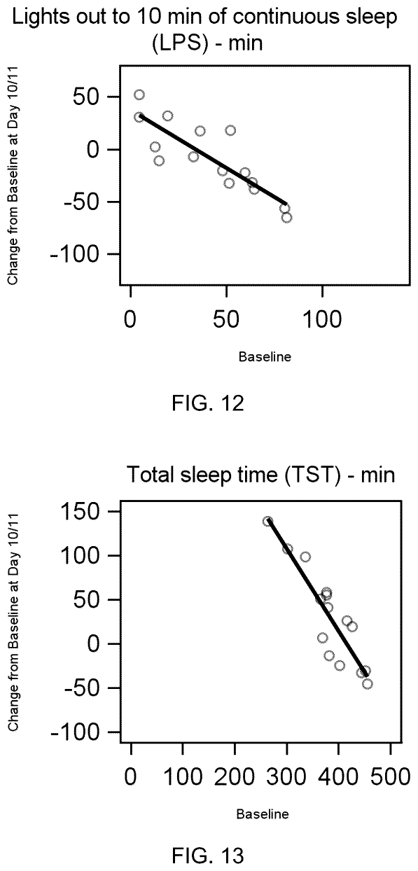

Compound A was found to be an orally active, selective antagonist of the orexin-2 receptor. After oral administration of 20 mg, Compound A had a short time to maximal plasma concentrations (Tₘₐₓ<1 hour) and was characterized by a short half-life (2-3 hours). Daytime administration of Compound A induced somnolence in healthy subjects while nighttime administration reduced the latency to persistent sleep (LPS) and prolongs the total sleep time (TST) in subjects with insomnia disorder (ID). The magnitude of the effect of Compound A on LPS and TST is directly related to level of insomnia at baseline. See, FIGS. 12-15.

Nighttime administration (within 30 minutes before bedtime) resulted in intermittent exposure of Compound A to plasma. Thus, it was demonstrated that repeated (10 days) daily dose administration did not result in accumulation.

### (ii) HDRS₁₇/HAM-D₆

A HDRS₁₇ total score was calculated by summing the 17 item scores taken during the study. A HDRS₁₇ total score ranges from 0 to 52, with higher scores indicating greater severity of depression. In order to correct for a possible effect of study medication on sleep, sleep-related items were removed from the HDRS₁₇ to calculate a (sleep item)-adjusted HDRS. Accordingly, an adjusted HDRS 17 total score was calculated by summing the item scores excluding the 3 insomnia questions (4-Insomnia Early, 5-Insomnia Middle and 6-Insomnia Late). A HDRS₁₇ adjusted total score ranges from 0 to 46. A 6-item subscale from the HDRS₁₇ (HAM-D₆) was analyzed and provided information to core depressive symptoms and is sensitive to treatment response. The six items included depressed mood, guilt feelings, work and interests, psychomotor retardation, psychic anxiety, and general somatics (tiredness and pains).

TABLE 7
Mean score ± standard deviation

| | | Compound | | |
|---|---|---|---|---|
| | | | Compound | |
| | | Placebo | A 20 mg | Diphenhydramine |
| Study | Time | (N = 12) | (N = 22) | 25 mg (N = 13) |
| Total | Baseline | 18.7 | 18.7 | 20.0 |
| HDRS₁₇ | | (5.71) | (4.65) | (5.12) |
| | Day 11 Change | −3.6 | −5.5 | −4.1 |
| | from baseline | (4.03) | (3.86) | (3.66) |
| Mean | Baseline | 13.7 | 14.4 | 15.1 |
| Adjusted | | (4.98) | (3.36) | (4.41) |
| HDRS17 | Day 11 Change | −2.3 | −4.5 | −2.3 |
| | from baseline | (3.03) | (2.76)* | (2.81) |

The results in Table 7 illustrate that the improvement in the total HDRS17 observed after administration of 20 mg of Compound A is mostly unrelated to changes in sleep (−5.5 versus −4.5 points) whereas sleep-related changes appear to more important for diphenhydramine (−4.1 versus −2.3).

The HAM-D6 score was calculated by summing the 6 items scores, and ranges from 0 to 22. Higher scores indicate greater severity of core symptoms.

TABLE 8
Mean scores ± standard deviation

| | | Compound | | |
|---|---|---|---|---|
| | | | Compound | |
| | | Placebo | A 20 mg | Diphenhydramine |
| Study | Time | (N = 12) | (N = 22) | 25 mg (N = 13) |
| mean HDRS₁₇ | Baseline | 4.3 | 4.8 | 5.1 |
| anxiety/ | | (1.56) | (1.56) | (1.80) |
| somatization | Day 11 | −0.8 | −1.6 | −0.9 |
| factor score | Change | (1.40) | (1.50) | (1.12) |
| | from | | | |
| | baseline | | | |
| Mean | Baseline | 9.0 | 10.4 | 10.6 |
| HAM-D₆ | | (3.57) | (2.09) | (3.31) |
| | Day 11 | −1.5 | −3.8 | −1.8 |
| | Change | (2.15) | (2.22)** | (2.01) |
| | from | | | |
| | baseline | | | |

The results in Table 8 illustrate that the change from baseline in the HDRS anxiety/somatization factor did not account for the observed improvement in depression ratings in the Compound A group. However, the core symptoms of depression (per HAM-D₆) did account for the observed improvement in depression ratings in the Compound A group.

### (iii) Polysomnography

The effects of study medication on polysomnography (PSG)-derived parameters was evaluated overnight on Days 1/2 and 10/11. In addition, PSG was recorded up to and following a forced wake overnight on Day 5/6. Two screening PSG recordings were made and baseline values were calculated as the average values recorded at Screening 1 and 2.

### (a) Total Sleep Time (TST)

TST is defined as total minutes spent in rapid eye movement (REM) and non-REM sleep. Compared to placebo, both Compound A and diphenhydramine increased TST overnight on Day 1/2. Because of an increase in TST in placebo-treated subjects on Day 10/11, the relative effect of Compound A and diphenhydramine were less pronounced. See, Table 9. Although the overall study population did not meet criteria for insomnia disorder (TST<360 minutes), individual subjects had baseline TST values as low as 263 minutes. Thus, the population was mixed with respect to the presence of insomnia disorder.

TABLE 9

| | Compound | | |
|---|---|---|---|
| | | Compound A | |
| Time | Placebo | 20 mg | Diphenhydramine |
| (min) | (N = 12) | (N = 22) | 25 mg (N = 13) |
| Baseline | 376 | 380 | 382 |
| | (56.2) | (50.1) | (47.2) |
| Day 1/2 Change | 7.4 | 30.9 | 28.3 |
| from baseline | (52.07) | (54.06) | (33.92) |
| Day 10/11 Change | 20.7 | 26.56 | 33.92 |
| from baseline | (64.44) | (56.11) | (46.01) |

The effect of Compound A on TST is proportional to the TST duration at baseline (FIG. 13). However, no relationship between the magnitude of the LPS change and the improvement in core depressive symptoms was observed (FIG. 15) supporting an antidepressant effect independent from an effect on insomnia.

### (b) Latency to Persistent Sleep

LPS is defined as the elapsed time (in minutes) from lights out to 10 minutes of continuous sleep. Compared to placebo, both Compound A and diphenhydramine modestly reduced LPS overnight on Day 1/2. Because of a decrease in LPS in placebo-treated subjects overnight on Day 10/11, the relative effect of Compound A and diphenhydramine were less pronounced. See, Table 10. Overall, the study population was characterized by a prolonged (>20 minutes) LPS. Similar as for TST, the population was mixed with respect to the presence of insomnia disorders at baseline with LPS values as low as 4.5 minutes.

TABLE 10

| | Compound | | |
|---|---|---|---|
| | | Compound A | |
| Time | Placebo | 20 mg | Diphenhydramine |
| (min) | (N = 12) | (N = 22) | 25 mg (N = 13) |
| Baseline | 53.8 | 40.9 | 36.0 |
| | (40.12) | (22.62) | (19.20) |
| Day 1/2 Change | 3.4 | 8.7 | 6.7 |
| from baseline | (46.39) | (36.04) | (26.40) |
| Day 10/11 Change | 17.5 | 9.2 | 0.3 |
| from baseline | (51.40) | (30.41) | (30.57) |

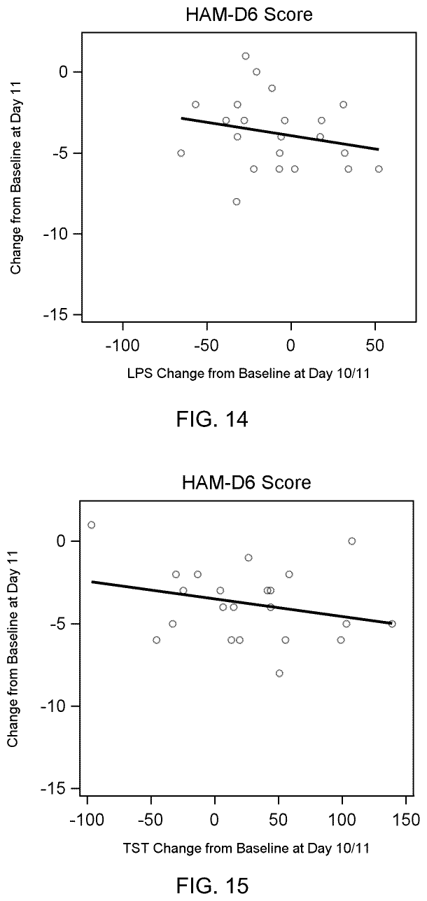

The effect of Compound A on LPS is proportional to the LPS duration at baseline (FIG. 12). However, no relationship between the magnitude of the LPS change and the improvement in core depressive symptoms was observed (FIG. 14) supporting an antidepressant effect independent from an effect on insomnia.

### (iv) Summary

These results show that, compared to placebo and diphenhydramine, the antidepressant effect of Compound A was larger and clinically relevant. Surprisingly, the effect of Compound A was largely related to an effect on the core symptoms of depression and overall unrelated to its effect on sleep related items. The antidepressant effect was sustained at least 14 days after treatment discontinuation. Of importance, improvements were already observed on Day 11 (first assessment) and were sustained upon treatment discontinuation.

### Example 3

This example was performed to illustrate that Compound A may be used in an adjunctive therapy. Specifically, Compound A was administered to subjects diagnosed with MDD (i) as a monotherapy and (ii) in combination with a known anti-depressant and the symptoms of depression of the subjects evaluated using the HDRS17 and HAM-D6 scale

In Group 1, thirty seven subjects were randomly assigned (in a 2:1:1 ratio) to 20 mg of Compound A, 25 mg diphenhydramine or placebo q.d. in the evening over 10 days. In Group 2, ten subjects were randomly assigned (in a 2:1:1 ratio) to 20 mg of Compound A, 25 mg diphenhydramine or placebo q.d. in the evening over 10 days. Each subject in Group 2 also took an amount of antidepressant selected from duloxetine, citalopram, paroxetine, or sertraline and as prescribed by their attending physician. For the evaluation of symptoms of depression for both groups, assessments were independently performed at screening and on Day 11, i.e., one day after the study, using HDRS₁₇ and HAM-D₆ as described in Example 2. The results of the evaluations are summarized in Tables 11 and 12. In Tables 11-12, # denotes the Cohen effect size, * denotes P<0.05 (statistically significant), and ** denotes P<0.01.

TABLE 11
Mean scores ± SD
Group I

| | | | Compound | |
|---|---|---|---|---|
| | | Placebo | A 20 mg | Diphenhydramine |
| Scale | Time | (N = 8) | (N= 18) | 25 mg (N = 11) |
| HDRS17 | Baseline | 19.5 (5.40) | 19.4 (4.80) | 21.5 (3.78) |
| | Day 11 | −2.5 (4.14) | −5.1 (3.97) | −4.2 (4.00) |
| | Change from | | | |
| | baseline | | | |
| | Effect Size | | −0.64 | |
| | Compound A | | | |
| | vs Placebo^{#} | | | |
| Adjusted | Baseline | 14.4 (4.50) | 14.9 (3.37) | 16.4 (3.35) |
| HDRS | Day 11 | −1.4 (3.29) | −4.3 (2.97)* | −2.1 (3.02) |
| | Change from | | | |
| | baseline | | | |
| | Effect Size | | −0.93 | |
| | Compound A | | | |
| | vs Placebo^{#} | | | |
| Anxiety- | Baseline | 4.4 (1.41) | 5.1 (1.53) | 5.5 (1.51) |
| Somatization | Day 11 | −0.8 (1.49) | −1.6 (1.65) | −0.8 (1.17) |
| Factor | Change from | | | |
| | baseline | | | |
| | Effect Size | | −0.51 | |
| | Compound A | | | |
| | vs Placebo^{#} | | | |
| HAM−D₆ | Baseline | 9.6 (3.70) | 10.8 (2.07) | 11.7 (2.05) |
| | Day 11 | −0.8 (2.25) | −3.6 (2.33)** | −1.7 (2.15) |
| | Change from | | | |
| | baseline | | | |
| | Effect Size | | −1.22 | |
| | Compound A | | | |
| | vs Placebo^{#} | | | |

TABLE 12
Group 2

| | | | Compound | |
|---|---|---|---|---|
| | | Placebo | A 20 mg | Diphenhydramine |
| | | (N = 4) | (N = 4) | 25 mg (N = 2) |
| HDRS17 | Baseline | 17.0 (6.78) | 15.5 (2.08) | 11.5 (0.71) |
| | Day 11 | −5.8 (3.20) | −7.0 (3.37) | −3.5 (0.71) |
| | Change from | | | |
| | baseline | | | |
| | Effect Size | | −0.37 | |
| | Compound A | | | |
| | vs Placebo^{#} | | | |
| | Baseline | 12.3 (6.29) | 11.8 (1.89) | 8.0 (1.41) |
| Adjusted | Day 11 | −4.3 (0.96) | −5.5 (1.29) | −3.5 (0.71) |
| HDRS | Change from | | | |
| | baseline | | | |
| | Effect Size | | −1.06 | |
| | Compound A | | | |
| | vs Placebo^{#} | | | |
| Anxiety- | Baseline | 4.3 (2.06) | 3.5 (1.00) | 2.5 (0.71) |
| Somatization | Day 11 | −1.0 (1.41) | −1.8 (0.50) | −1.5 (0.71) |
| Factor | Change from | | | |
| | baseline | | | |
| | Effect Size | | −0.76 | |
| | Compound A | | | |
| | vs Placebo^{#} | | | |
| HAM−D₆ | Baseline | 7.8 (3.40) | 8.8 (1.26) | 4.5 (0.71) |
| | Day 11 | −3.0 (0.82) | −4.8 (1.50) | −2.0 (1.41) |
| | Change from | | | |
| | baseline | | | |
| | Effect Size | | −1.49 | |
| | Compound A | | | |
| | vs Placebo^{#} | | | |

These results illustrate that Compound A has antidepressant efficacy in untreated and antidepressant drug-treated subjects with MDD supporting its efficacy as monotherapy and adjunctive therapy.

The disclosures of each patent, patent application, and publication cited or described in this document are hereby incorporated herein by reference, in its entirety.

Those skilled in the art will appreciate that numerous changes and modifications can be made to the preferred embodiments of the disclosure and that such changes and modifications can be made without departing from the spirit of the disclosure. It is, therefore, intended that the appended claims cover all such equivalent variations as fall within the true spirit and scope of the disclosure.

What is claimed is:

1. A method of treating major depressive disorder in a human patient, comprising administering to the patient in need thereof about 20 mg of a compound that is [5-(4,6-dimethyl-pyrimidin-2-yl)hexahydro-pyrrolo[3,4-c]pyrrol-2-yl]-(2-fluoro-6-[1,2,3]triazol-2-yl-phenyl)-methanone: 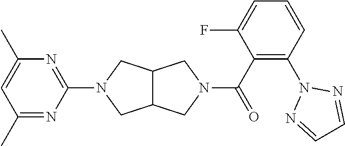 [SMILES: Cc1cc(C)nc(N2CC3CN(C(=O)c4c(F)cccc4-n4nccn4)CC3C2)n1]
or a pharmaceutically acceptable salt thereof, wherein the compound is administered once daily and prior to sleep, and wherein the compound treats a psychic symptom of the depression.

2. The method of claim 1, wherein the compound is administered at night.

3. The method of claim 2, wherein antidepressant effect from the compound is maintained when the patient is awake the next day.

4. The method of claim 1, wherein the compound is [5-(4,6-dimethyl-pyrimidin-2-yl)hexahydro-pyrrolo[3,4-c]pyrrol-2-yl]-(2-fluoro-6-[1,2,3]triazol-2-yl-phenyl)-methanone.

5. The method of claim 1, wherein the compound [5-(4,6-dimethyl-pyrimidin-2-yl)hexahydro-pyrrolo[3,4-c]pyrrol-2-yl]-(2-fluoro-6-[1,2,3]triazol-2-yl-phenyl)-methanone hydrochloride.

6. The method of claim 1, wherein the compound is a hydrate of the pharmaceutical salt.

7. The method of claim 1, wherein the compound is a hydrate of [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone hydrochloride.

8. The method of claim 1, wherein the once daily administration is repeated.

9. The method of claim 8, wherein the compound is administered for at least four weeks.

10. The method of claim 1, wherein the compound is administered in the form of a tablet.
